(19) United States
(12) Patent Application Publication
(10) Pub. No.: US 2014/0276161 A1
(43) Pub. Date: Sep. 18, 2014

(54) METHOD AND APPARATUS FOR DISPLAYING PERIODIC SIGNALS GENERATED BY A MEDICAL DEVICE

(71) Applicant: GestInTime, Inc., Palo Alto, CA (US)

(72) Inventor: Suresh SUBRAMANIAM, Palo Alto, CA (US)

(73) Assignee: GestlnTime, Inc., Palo Alto, CA (US)

(21) Appl. No.: 13/838,563

(22) Filed: Mar. 15, 2013

(51) Int. Cl. A61B 5/00; A61B 5/0476; A61B 5/0488; A61B 5/044

(52) U.S. Cl. CPC A61B 5/7445; A61B 5/044; A61B 5/0476; A61B 5/0488
USPC 600/523; 600/300; 600/544; 600/546

(57) ABSTRACT

An improved method and apparatus for displaying periodic signals generated by a medical device is disclosed.

### TECHNICAL FIELD

An improved method and apparatus for displaying periodic signals generated by a medical device is disclosed.

### BACKGROUND OF THE INVENTION

Electrocardiogram (EKG, also known as ECG) devices are well-known in the prior art. They measure the electrical activity of the human heart using electrodes and create tracings of the activity on paper or on a visual display.

FIG. 1 depicts a prior art medical device 10 along with output 20. In this particular exemplary depiction, medical device 10 is an EKG device and output 20 is EKG data. Notably, output 20 comprises either a graph printed on a scroll of paper or a graphical display on a screen that scrolls in real-time as the electrical activity is measured. Using prior art device 20, a doctor or medical professional must read the scroll of paper or watch the tracings on a screen in real time. This can be a tedious and challenging exercise that contains the inherent risk that the doctor or medical professional will miss an important change in the monitored activity.

Many medical devices create periodic signals as well that represent activity within the human body. For example, medical devices exist in the areas of electromyography (EMG) (to monitor muscle activity), electroencephalography (EEG) (to monitor brain activity), polysomnography (to monitor breathing activity during sleep), and other areas in which periodic signals are generated and monitored in real-time by a doctor or medical professional.

In the electrical engineering field, oscilloscopes and other tools are well-known for displaying electrical signals on a screen. One technique used by such tools is to create an “eye diagram” for periodic signals. The technique involves superimposing the signal from one period over the signal from the next period and the next period, and so on. An exemplary eye diagram 30 is shown in FIG. 2. This allows the user to physically see multiple periods of the signal at one time in a limited amount of space and to readily view any differences or deviations in the signals.

What is needed is a device for generating an eye diagram for periodic signals generated by medical devices and to identify any excursions from the mean values, expected values, or other thresholds.

### SUMMARY OF THE INVENTION

The aforementioned problem and needs are addressed through an embodiment for generating an eye diagram of a periodic signal output from a medical device.

### BRIEF DESCRIPTION OF THE DRAWINGS

FIG. 1 depicts a prior art medical device and its output.

FIG. 2 depicts a prior art eye diagram.

FIG. 3 depicts an embodiment for generating an eye diagram using a periodic signal from a medical device.

FIG. 4 depicts an embodiment for generating an eye diagram using a periodic signal where the eye diagram shows an excursion in the signal.

FIG. 5 depicts an embodiment for displaying an expanded version of the periodic signal in response to a user instruction after viewing the eye diagram of FIG. 4.

FIG. 6 depicts various display options for the eye diagram.

FIG. 7 depicts an embodiment of display eyewear.

### DETAILED DESCRIPTION OF THE PREFERRED EMBODIMENTS

An embodiment will now be described with reference to FIG. 3. Medical device 10 is the same prior art device described previously with reference to FIG. 1. The output of medical device 10 is provided as an input to processing device 40. In this particular example, the output is EKG data, but the same principles apply to any periodic data collected from a medical device.

In one embodiment, processing device 40 is a computing device (such as a desktop, notebook, server, tablet, mobile device, or other computer) comprising a processor, memory, I/O connection (such as a USB connection) for communicating with a medical device, and an I/O connection for sending output to a display, printer, or other device. Optionally, processing device 40 can itself include a display (as might be the case if processing device 40 is a tablet or mobile device). Processing device 40 comprises software code for performing the functions described herein.

Processing device 40 receives the periodic signal generated by medical device 10 and generates an output 50 that comprises an eye diagram of the signal by superimposing one period of the signal on top of another period of the signal, and so on. One of ordinary skill in the art will appreciate that output 50 is much easier to read and analyze than output 20 shown in FIG. 1.

A further example is shown in FIG. 4. FIG. 4 is similar to FIG. 3 except output 50 shows an excursion 60 in one period of the signal. This represents a deviation from the “norm” as shown in the eye diagram. One of ordinary skill in the art will understand that excursion 60 is much easier to identify than it would have been in the traditional tracings on a scroll of paper or tracings displayed on a screen that scrolls in real-time.

Optionally, processing device 40 can generate alert 70. Alert 70 can appear on the display as part of output 50, or it can be sent over email, SMS or MMS message, a phone call, a web-based message, etc. Processing device 40 can generate alert 70 based on any of the following: statistically significant deviation from the mean value of the periodic signal at that location within the period; significant deviation from the expected value of the signal for a healthy individual; or a value above a pre-determined threshold specified by the user or programmed into processing device 40.

Optionally, processing device 40 can enable a user to request more information regarding excursion 60 or any other portion of the eye diagram contained in output 50. Such requests can be made through a mouse click on a display, through a keyboard, or using a voice command.

If a user requests further information regarding excursion 60 (such as by clicking on it using a mouse and a display), then optionally a traditional view will be created as shown in FIG. 5.

In FIG. 5, processing device 40 generates output 70, which resembles a traditional display of periodic signal. Excursion 60 is shown, and the selected period 70 in which excursion 60 appears is highlighted for the user, such as by drawing a box around the relevant portion of the signal as shown in FIG. 5, altering the color or brightness of that portion of the signal, or otherwise changing the appearance of that portion of the signal.

One of ordinary skill in the art will understand that this combination of the prior art medical devise with the prior art eye diagram technique yields an invention that will enhance the ability of doctors and other medical professionals to analyze periodic signal from medical devices, such as EKG or ECG data, and to quickly identify any troublesome excursions in the data.

FIG. 6 depicts various mechanisms for a user to view output 70. These mechanisms include a display 100 (such as an LCD), mobile device 110 (such as a tablet or mobile phone), and eyewear 120.

FIG. 7 depicts an example of eyewear 120. Eyewear 120 comprises lenses 122 and frame 121 (just as with normal glasses). But it also includes display unit 130 and processing and transmission unit 140 (embedded within the frame 121).

An example of eyewear 120 was recently announced by Google as the “Google Glass” product. Eyewear 120, such as the Google Glass, comprises a display unit 130 that displays data that you could otherwise display on an LCD or other display. Display unit 130 can be used to display the eye diagrams discussed previously.

The possible uses of eyewear 120 by physicians in conjunction with the display of periodic signals discussed above are numerous. For example, a physician could: (a) view a periodic signal during a patient examination, during a remote consultation, or during a collaborative session with a fellow physician (e.g., two physicians viewing the same EKG); (b) look at the patient in the physician's office while the display unit 130 displays a periodical signal; or (c) apply physical pressure to the patient or perform other techniques or tests and get instant visual feedback regarding the effect on heartbeat, etc.

References to the present invention herein are not intended to limit the scope of any claim or claim term, but instead merely make reference to one or more features that may be covered by one or more of the claims. Materials, processes and numerical examples described above are exemplary only, and should not be deemed to limit the claims. It should be noted that, as used herein, the terms “over” and “on” both inclusively include “directly on” (no intermediate materials, elements or space disposed there between) and “indirectly on” (intermediate materials, elements or space disposed there between). Likewise, the term “adjacent” includes “directly adjacent” (no intermediate materials, elements or space disposed there between) and “indirectly adjacent” (intermediate materials, elements or space disposed there between). For example, forming an element “over a substrate” can include forming the element directly on the substrate with no intermediate materials/elements there between, as well as forming the element indirectly on the substrate with one or more intermediate materials/elements there between.

What is claimed is:

1. A system for processing and displaying a periodic signal from a medical device, comprising:
a medical device for generating a periodic signal;
a processing device for receiving the periodic signal and generating an eye diagram based on the periodic signal;
a display coupled to the processing device for displaying the eye diagram.

2. The system of claim 1, wherein the medical device is an electrocardiogram device.

3. The system of claim 1, wherein the medical device is an electroencephalography device.

4. The system of claim 1, wherein the medical device is an electromyography device.

5. The system of claim 1, wherein the processing device is configured to generate an alert if the received data at a location within a period of the periodic signal exceeds the mean of prior data at that location.

6. The system of claim 1, wherein the processing device is configured to generate an alert if the received data at a location within a period of the periodic signal exceeds a pre-determined threshold.

7. The system of claim 1, wherein the processing device is capable of receiving a request from a user relating to the periodic signal.

8. The system of claim 7, wherein the processing device is capable of highlighting a portion of the periodic signal in response to the request.

9. The system of claim 1, wherein the display is contained within eyewear.

10. The system of claim 9, wherein the display is contained within a tablet.

11. A method for displaying a periodic signal from a medical device, comprising:
receiving a periodic signal from a medical device;
generating an eye diagram based on the periodic signal; and
displaying the eye diagram on a display.

12. The method of claim 11, wherein the medical device is an electrocardiogram device.

13. The method of claim 11, wherein the medical device is an electroencephalography device.

14. The method of claim 11, wherein the medical device is an electromyography device.

15. The method of claim 11, further comprising generating an alert if the received data at a location within a period of the periodic signal exceeds the mean of prior data at that location.

16. The method of claim 11, further comprising generating an alert if the received data at a location within a period of the periodic signal exceeds a pre-determined threshold.

17. The method of claim 11, further comprising receiving a request from a user relating to the periodic signal.

18. The method of claim 17, wherein the processing device is capable of highlighting a portion of the periodic signal in response to the request.

19. The method of claim 11, wherein the display is contained within eyewear.

20. The method of claim 11, wherein the display is contained within a tablet.
